(19) United States
(12) Patent Application Publication
(10) Pub. No.: US 2026/0308667 A1
(43) Pub. Date: Oct. 8, 2026

(54) DELAYED SIGNAL MELT PROBES AND USES THEREOF

(71) Applicant: Luminex Corporation, Austin, TX (US)

(72) Inventors: Kyle KNIGHT, Austin, TX (US); Debora FERRI, Austin, TX (US)

(73) Assignee: Luminex Corporation, Austin, TX (US)

(21) Appl. No.: 19/637,458

(22) Filed: Apr. 2, 2026

Related U.S. Application Data
(60) Provisional application No. 63/782,790, filed on Apr. 3, 2025.

(51) Int. Cl. C12Q 1/6825; C12Q 1/6806; C12Q 1/6823; C12Q 1/686

(52) U.S. Cl. CPC C12Q 1/6825; C12Q 1/6806; C12Q 1/6823; C12Q 1/686; C12Q 2600/16

(57) ABSTRACT

Provided herein are methods of detecting at least two target nucleic acids in a sample, comprising providing two or more probes per channel during PCR, wherein the first high temperature probe has a melting temperature (Tₘ) for a first amplicon target that is above the set annealing temperature of the reaction, and the second low temperature probe has a melting temperature that is below the annealing temperature of the reaction. Detecting the first target may be accomplished by measuring the signal from the first high temperature probe at the annealing temperature of PCR cycles during PCR. Detecting the second target may be accomplished by measuring the signal from the first low temperature probe at more than one temperature after PCR and detecting the presence of the second target by a change in signal of the first low temperature probe over more than one temperature during a post-PCR melt analysis. Also provided herein are kits and compositions for detecting at least two target nucleic acids in a sample.

### REFERENCE TO RELATED APPLICATIONS

The present application claims the priority benefit of U.S. provisional application No. 63/782,790, filed Apr. 3, 2025, the entire contents of which are incorporated herein by reference.

### REFERENCE TO A SEQUENCE LISTING

This application contains a Sequence Listing XML, which has been submitted electronically and is hereby incorporated by reference in its entirety. Said Sequence Listing XML, created on Apr. 2, 2026, is named LUMNP0169US.xml and is 2,625 bytes in size.

### BACKGROUND

### 1. Field

The present disclosure relates generally to the field of molecular biology. More particularly, it concerns methods and compositions for identifying nucleic acid targets in biological samples.

### 2. Description of Related Art

Polymerase chain reaction (PCR) is a molecular biology technique for enzymatically replicating DNA without using a living organism. PCR is commonly used in medical and biological research labs for a variety of tasks, such as, for example, the detection of hereditary diseases, the identification of genetic fingerprints, the diagnosis of infectious diseases, the cloning of genes, paternity testing, and DNA computing. PCR may be used for nucleic acid detection because of its unparalleled amplification and precision capability. DNA detection is typically performed at the endpoint, or plateau phase of the PCR reaction, making it difficult to quantify the starting template. Real-time PCR or kinetic PCR advances the capability of end-point PCR analysis by recording the amplicon concentration as the reaction progresses. Amplicon concentration may be recorded by a fluorescent signal change associated with the amplified target. Real-time PCR may also be advantageous over end-point detection in that contamination is limited because it can be performed in a closed system. Other advantages may include greater sensitivity, dynamic range, speed, and fewer processes required.

Several assay chemistries have been used in real-time PCR detection methods. These assay chemistries may include using double-stranded DNA binding dyes, dual-labeled oligonucleotides, such as hairpin primers, and hairpin probes. However, a drawback of current real-time PCR is its limited multiplexing capability. Some current real-time PCR technologies use reporter fluorochromes that are free in solution. This design necessitates the use of spectrally distinct fluorochromes for each assay within a multiplex reaction. For example, a multiplex reaction designed to detect 4 target sequences would require an instrument capable of distinguishing 4 different free floating fluorochromes by spectral differentiation, not including controls. These requirements not only limit the practical multiplexing capability, but also increase costs since such instruments typically require multiple emission sources, detectors, and filters. Current real-time PCR technologies may have multiplexing capabilities from about 1-6 plex.

Certain sample processing techniques, including for example PCR and related processing, utilize thermal cycling by sequentially increasing and decreasing the temperature of the sample for a number of cycles. Particular techniques may employ a significant number of cycles in which the temperature of the sample is maintained within maximum and minimum temperature ranges for specified periods of time during each cycle. Processes utilizing a large number of cycles and extended periods of time within or transitioning between each cycle can take an extended amount of time to complete. Reducing the amount of time needed to transition the sample between the maximum and minimum temperature ranges (and vice versa) can reduce the overall time required to thermally cycle the sample. Reducing the thermal cycling time can reduce the overall sample processing time and provide for more efficient sample processing.

Thermal cyclers are limited in the number of optical channels that can be used to interrogate specific nucleic acid targets with each channel typically only able to detect a single target. Many thermal cyclers will have 4 or 5 optical channels limiting the number of multiplexable targets to 4 or 5. Distinguishing targets by melt analysis is known in the art as a method for increasing the multiplex of the assay within each channel. This is usually done as an end-point analysis once the PCR cycling procedure has ended. There is a need in some cases to detect one nucleic acid target in real-time, but use the end point analysis to detect a second target nucleic acid without the second target signal interfering with the real-time signal of the first target.

### SUMMARY

Provided herein are methods of detecting at least two target nucleic acids in a sample, the method comprising the steps of:
a) contacting the sample with reagents comprising:
   i) a first set of primers for PCR amplification of a first target nucleic acid, with a first melting temperature;
   ii) a second set of primers for PCR amplification of a second target nucleic acid, with a second melting temperature;
   iii) a first high temperature probe, wherein the first high temperature probe hybridizes to at least a portion of the first target nucleic acid and produces a first signal after binding thereto, and wherein the first high temperature probe has a third melting temperature that is above the first and second melting temperatures;
   iv) a first low temperature probe, wherein the first low temperature probe hybridizes to at least a portion of the second target nucleic acid and produces a second signal after binding thereto, wherein the second signal is identical to the first signal, and wherein the first low temperature probe has a fourth melting temperature that is below the first and second melting temperatures;
b) amplifying the first and second target nucleic acids by a PCR reaction, wherein the PCR comprises at least two thermocycles at temperatures greater than the fourth melting temperature, and wherein the second signal is not produced during the PCR reaction;
c) detecting the first target nucleic acid by detecting the first signal from the high temperature probe during at least two thermocycles of the PCR reaction;
d) detecting the presence of the second target nucleic acid by detecting the second signal from the first low temperature probe over at least two temperatures during a post-PCR melt analysis, wherein at least one of the temperatures is below the fourth melting temperature.

The first melting temperature and the second melting temperature may be the same or may be less than 5° C., 4° C., 3° C., 2° C., 1° C., or 0.5° C. different from each other. The first melting temperature and the second melting temperature may be less than 10%, 9%, 8%, 7%, 6%, 5%, 4%, 3%, 2%, or 1% different from each other.

The third melting temperature may be about 3° C., 4° C., 5° C., 6° C., 7° C., 8° C., 9° C., 10° C., 11° C., 12° C., 13° C., 14° C., or 15° C. higher than the first melting temperature. The third melting temperature may be about 5° C., 6° C., 7° C., 8° C., 9° C., 10° C., 11° C., 12° C., 13° C., 14° C., or 15° C. higher than the fourth melting temperature.

The fourth melting temperature may be about 3° C., 4° C., 5° C., 6° C., 7° C., 8° C., 9° C., 10° C., 11° C., 12° C., 13° C., 14° C., or 15° C. lower than the first melting temperature.

The first high temperature probe and/or the first low temperature probe may further comprise a fluorescent label, a fluorescent quencher, a minor groove binder, a hairpin, one or more internal nucleotide labelled fluorescent dyes, and/or a cleavable probe.

The first and/or second signals from the first high temperature probe and/or first low temperature probe may be fluorescent signal(s).

The first high temperature probe may be a TaqMan probe.

The PCR reaction of the present methods may amplify a short fragment of the first and second nucleic acids. The PCR reaction may be completed in less than 20 minutes. The PCR reaction may comprise a high delta cycling phase and/or a low delta cycling phase. The high delta cycling phase may comprise annealing temperatures at 58° C. for 30 second holds, and denaturing temperatures at 105° C. for 1 second holds. The low delta cycling phase may comprise annealing temperatures at 70° C. for 30 second holds, and denaturing temperatures at 106° C. for 1 second holds.

The post-PCR melt analysis may comprise thermocycling steps starting at 47° C. and ending at 61° C. with 1° C. increments and 20 second holds at each step. The thermocycling steps may comprise at least one temperature below the fourth melting temperature, and at least one temperature above the fourth melting temperature.

The sample may further comprise a third and fourth target nucleic acid, and wherein step a) may further comprise contacting the sample with:
v) a third set of primers for PCR amplification of the third target nucleic acid, with a fifth melting temperature;
vi) a fourth set of primers for PCR amplification of the fourth target nucleic acid, with a sixth melting temperature;
vii) a second high temperature probe wherein the second high temperature probe hybridizes to at least a portion of the third target nucleic acid and produces a third signal, and wherein the second high temperature probe has a seventh melting temperature that is above the first, second, fifth, and sixth melting temperatures;
viii) a second low temperature probe, wherein the second low temperature probe hybridizes to at least a portion of the fourth target nucleic acid and produces a fourth signal, wherein the fourth signal is identical to the third signal, and wherein the second low temperature probe has a eighth melting temperature that is below the first, second, fifth, and sixth melting temperatures;
   and wherein step b) may further comprise amplifying the third and fourth target nucleic acids by a PCR reaction, wherein the PCR may comprise at least two thermocycles at temperatures greater than the fourth and eighth melting temperatures, and wherein the second and fourth signals may not be produced during the PCR reaction; and wherein step c) may further comprise detecting the first and third target nucleic acids by detecting the first and third signals from the first and second high temperature probes during at least two thermocycles of the PCR reaction; and wherein step d) may further comprise detecting the presence of the second and fourth target nucleic acids by detecting the second and fourth signals from the first and second low temperature probes over at least two temperatures during a post-PCR melt analysis, wherein at least one of the temperatures is below the fourth and eighth melting temperatures.

The second high temperature probe and/or the second low temperature probe may further comprise a fluorescent label, a fluorescent quencher, a minor groove binder, a hairpin, a cleavable probe, and/or one or more internal nucleotide labelled fluorescent dyes.

The second and/or third signals from the second high temperature probe and/or second low temperature probe may be fluorescent signal(s).

The second high temperature probe may be a TaqMan probe.

The PCR reaction may amplify a short fragment of the second and third nucleic acids. The PCR reaction may be completed in less than 20 minutes.

Provided herein are kits comprising:
a) a first set of primers for PCR amplification of a first target nucleic acid, with a first melting temperature;
b) a second set of primers for PCR amplification of a second target nucleic acid, with a second melting temperature;
c) a first high temperature probe, wherein the first high temperature probe hybridizes to at least a portion of the first target nucleic acid and produces a first signal, and wherein the first high temperature probe has a third melting temperature that is above the first and second melting temperatures;
d) a first low temperature probe, wherein the first low temperature probe hybridizes to at least a portion of the second target nucleic acid and produces a second signal, wherein the second signal is identical to the first signal, and wherein the first low temperature probe has a fourth melting temperature that is below the first and second melting temperatures.

Provided herein are compositions comprising:
a) a first high temperature probe, wherein the first high temperature probe hybridizes to at least a portion of a first target nucleic acid and produces a first signal, and wherein the first high temperature probe has a first melting temperature;
b) a first low temperature probe, wherein the first low temperature probe hybridizes to at least a portion of a second target nucleic acid and produces a second signal, wherein the second signal is identical to the first signal, and wherein the first low temperature probe has a second melting temperature that is below the first melting temperature.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 depicts an exemplary temperature cycling profile of a polymerase chain reaction wherein cycles 6 through 50 oscillate between a temperature of 65° C. and 105° C. A low temperature probe with a Tₘ of 57° C. and a high temperature probe with a Tₘ of 72° C. are present in the reaction. The inverse derivative of the signal from multiple reads over many temperatures is graphed and identifies the specific Tₘ of the low temperature probe, adding a confirmation in specificity.

FIG. 2 depicts an exemplary PCR cycling and melt profile. An initial 2 min 75° C. Lysis step followed by a 5 min 48° C. hold for reverse transcription and a 2 min 94° C. hold for hot start of the DNA polymerase enzyme. The PCR cycling parameters in this experiment contain 2 stages, high delta (HD) cycling and low delta cycling (LD). The first 5 rounds of thermal cycling are high delta, with annealing temperatures at 58° C. for 30 sec holds and the instrument heated to a denature temperature of 105° C. for 1 sec hold. The remaining 40 PCR cycles are low delta with an annealing temp of 70° C. for 30 sec holds and a denature temperature of 106° C. for 1 sec hold. Post PCR cycling, a probe melt step was performed starting at 47° C. and ending at 61° C. with 1° C. increments and 20 sec holds at each step.

FIG. 3 depicts exemplary data from 40 PCR cycles and post melt normalized to step 17. Flu A was tested at 10×LOD with and without MS2 target present. One line in the graph shows positive amplification during PCR representing Flu A positivity with MS2 present. One line on the graph shows the same 10×LOD Flu A positivity during PCR but since no MS2 target was present there is not a drastic signal change from the MS2 probe. Another line in the graph is a Flu A negative with MS2 positive sample. The graph also shows a Flu A and MS2 negative sample.

FIG. 4 depicts the derivative of the normalized data from FIG. 3. The change in signal from the first data point is calculated to baseline all the data to 0. Lines that correspond to MS2 negative samples do not show a derivative peak compared to the positive MS2 samples.

### DETAILED DESCRIPTION

Polymerase Chain Reaction (PCR) is a technique for amplifying DNA without a living organism, widely used in medical and biological research for tasks like detecting hereditary diseases, genetic fingerprinting, diagnosing infectious diseases, cloning genes, paternity testing, and DNA computing. Real-time PCR enhances traditional PCR by measuring DNA amplification in real-time, offering benefits like reduced contamination, greater sensitivity, and faster results. However, it faces challenges with multiplexing due to the need for distinct fluorochromes, increasing costs and complexity.

The present disclosure provides methods of detecting more than one nucleic acid target per channel comprising, providing two or more probes per optical channel during PCR, wherein the first high temperature probe has a melting temperature (Tₘ) for a first amplicon target that is above the set annealing temperature of the reaction, and the first low temperature probe has a melting temperature that is below the annealing temperature of the reaction. Detecting the first target may be accomplished by measuring the signal of the first high temperature probe at the annealing temperature of PCR cycles during PCR. Detecting the second target may be accomplished by measuring the signal of the first low temperature probe at more than one temperature after PCR and detecting the presence of the second target by a change in signal of the first low temperature probe over more than one temperature during a post-PCR melt analysis. The first low temperature probe does not produce signal during PCR, but does produce signal after PCR, because the range of temperatures interrogated during the post-PCR melt analysis phase of the reaction is at least partly within the window of temperatures that would produce a change in signal. The first high temperature probe may be capable of producing signal during PCR, the first low temperature probe may not be capable of producing signal during PCR due to the set point of the interrogation or annealing temperature being well above the melting temperature of the second probe.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### II. SAMPLES

As used herein, the term “sample” generally refers to any material containing or suspected of containing a nucleic acid. A sample may include a bodily tissue or a bodily fluid including but not limited to blood (or a fraction of blood, such as plasma or serum), lymph, mucus, tears, urine, and saliva. A sample may comprise DNA (e.g., genomic DNA), RNA (e.g., mRNA), and/or cDNA, any of which may be amplified to provide an amplified nucleic acid. A sample may comprise material obtained from an environmental locus (e.g., a body of water, soil, and the like) or material obtained from a fomite (i.e., an inanimate object that serves to transfer pathogens from one host to another).

As used herein “nucleic acid” generally refers to a polymeric form of nucleotides of any length (e.g. at least 2, 3, 4, 5, 6, 10, 50, 100, 200, 500 or 1000 nucleotides), either deoxyribonucleotides or ribonucleotides or a combination thereof, and any modifications thereof. Modifications include, but are not limited to, those that provide other chemical groups that incorporate additional charge, polarizability, hydrogen bonding, electrostatic interaction, and fluxionality to the nucleic acid ligand bases or to the nucleic acid ligand as a whole. Accordingly, the nucleic acids described herein include not only the standard bases adenine (A), cytosine (C), guanine (G), thymine (T), and uracil (U) but also non-standard or non-natural nucleotides, analogs and derivatives thereof. Non-standard or non-natural nucleotides such as isoC or isoG, are described, for example, in U.S. Pat. Nos. 5,432,272, 5,965,364, 6,001,983, 6,037,120, and 6,140,496, all of which are incorporated herein by reference and include bases other than A, G, C, T, or U that can be incorporated into a growing nucleic acid strand by a polymerase and are capable of base-pairing with a complementary non-standard or non-natural nucleotide to form a base pair.

As used herein, “target,” “target sequence” or “target nucleic acid” refers to a nucleic acid sequence of interest. A “target,” “target sequence” or “target nucleic acid” may also be a surrogate nucleic acid sequence that is formed in the presence of a nucleic acid sequence of interest in a sample and is representative of the nucleic acid sequence of interest. The surrogate nucleic acid sequence may be an amplification product or a cleavage product that is generated upon specific binding to a nucleic acid sequence of interest.

### III. POLYMERASE CHAIN REACTION (PCR)

Provided herein are methods of detecting at least two target nucleic acids in a sample, the method comprising amplification of target nucleic acids in a sample, and detection of at least a first target nucleic acid during the PCR reaction, and detection of at least a second target nucleic acid during a post-PCR melt analysis.

As used herein, “amplification” or “amplifying” refers to the production of additional copies of a nucleic acid sequence or a target sequence. Amplification may be carried out using polymerase chain reaction (PCR) or other amplification technologies known in the art, such as isothermal amplification. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, nucleoside triphosphates, and optionally, at least one labeled probe and/or optionally, at least one agent for determining the melting temperature of an amplified target nucleic acid (e.g., a fluorescent intercalating agent that exhibits a change in fluorescence in the presence of double-stranded nucleic acid). One or more primers or probes in the amplification mixture may be labeled with a reporter that emits a detectable signal (e.g., a fluorophore). The amplification mixture may include at least one nucleotide that is labeled with a quencher (e.g., Dabcyl). The probe may include both a fluorophore and a quencher.

An oligonucleotide is a nucleic acid that includes at least two nucleotides. An oligonucleotide may be designed to function as a “primer.” A “primer” is a short nucleic acid, usually a ssDNA oligonucleotide, which may be hybridized to a target nucleic acid by complementary base-pairing. The primer may then be extended along the target nucleic acid template strand by a polymerase enzyme, such as a DNA polymerase enzyme or an RNA polymerase enzyme. Primer pairs can be used for amplification (and identification) of a target nucleic acid sequence (e.g., by the polymerase chain reaction (PCR)).

A primer that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. “Specific hybridization” is an indication that two nucleic acid sequences share a high degree of complementarity, and two nucleic acid strands that specifically hybridize are said to be “complementary.” Specific hybridization complexes form under permissive annealing conditions. Permissive conditions for annealing of nucleic acid sequences are routinely determinable by one of ordinary skill in the art and may occur, for example, at 65° C. in the presence of about 6×SSC. Annealing temperatures are typically selected to be about 5° C. to 20° C. lower than the thermal melting point T_{M} for the specific sequence at a defined ionic strength and pH. The T_{M} is the temperature (under defined ionic strength and pH) at which 50% of the target sequence hybridizes to a perfectly matched probe. Equations for calculating T_{M}, for example, nearest-neighbor parameters, and conditions for nucleic acid hybridization, are known in the art.

Real-time polymerase chain reaction may be employed in the instant methods. A real-time polymerase chain reaction (real-time PCR, or qPCR) is a laboratory technique of molecular biology based on the polymerase chain reaction (PCR). It monitors the amplification of a targeted DNA molecule during the PCR (i.e., in real time), not at its end, as in conventional PCR. Real-time PCR can be used quantitatively and semi-quantitatively (i.e., above/below a certain amount of DNA molecules). Two exemplary methods for the detection of PCR products in real-time PCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence. Several assay chemistries have been used in real-time PCR detection methods. These assay chemistries include using double-stranded DNA binding dyes, dual-labeled oligonucleotides, such as hairpin primers, and hairpin probes.

Digital polymerase chain reaction may be used in the instant methods. As used herein, “digital polymerase chain reaction (dPCR)” refers to an amplification reaction where a sample is separated into a large number of partitions and the reaction is carried out in each partition individually. dPCR involves partitioning the sample such that individual nucleic acid molecules contained in the sample are localized in many separate regions, such as in individual wells in microwell plates or micropartitions, in the dispersed phase of an emulsion, or arrays of nucleic acid binding surfaces. Ideally, each partition will contain 0 or 1 copy of each target nucleic acid, providing a negative or positive reaction, respectively. Unlike conventional PCR, dPCR is not dependent on the number of amplification cycles to determine the initial amount of the target nucleic acid in the sample. Accordingly, dPCR eliminates the reliance on exponential data to quantify target nucleic acids and provides absolute quantification. Bead emulsion PCR, which clonally amplifies nucleic acids on beads in an emulsion, is one example of a dPCR technique in which the reactions are portioned into droplets. See, e.g., U.S. Pat. Nos. 8,048,627 and 7,842,457, which are hereby incorporated by reference. When dPCR is performed in an emulsion, the emulsion should be heat stable to allow it to withstand thermal cycling conditions. In contrast, devices designed to form compartments or partitions in a static array on a planar surface may be used to perform dPCR. For example, WO2018094091, incorporated herein by reference, describes a device that directs fluids into partitions and subsequently isolates partitions from each other. Several other ways of forming static arrays or reaction chambers are have been described, for example as in U.S. Pat. Nos. 9,039,993, 9,643,178 PCT/US2003/041356, EP2906348 and Du et. al. “SlipChip” Lab on a Chip 9 (16): 2286.

The nucleic acids disclosed herein may be prepared by any technique known to one of ordinary skill in the art, such as for example, chemical synthesis, enzymatic production, or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al., 2001, incorporated herein by reference).

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA (cDNA).

A reverse transcriptase PCR™ amplification procedure may be performed to reverse transcribe mRNA into cDNA. Methods of RT-PCR are well known in the art (see Sambrook et al., 2001). Alternative methods for RT-PCR utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of nucleic acid sequences that may be used in the practice of certain aspects of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids, which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA).

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR™” (Frohman, 1990; Ohara et al., 1989).

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

### IV. MELT ANALYSIS

Methods provided herein comprise detection of at least a second target nucleic acid in a sample by a post-PCR melt analysis by detecting a first low temperature probe in a sample . . .

Melt analysis refers to a process by which hybridization between complementary strands of a nucleic acid duplex is reversed and changes in signal are monitored as the strands dissociate. Starting at or below a temperature at which the duplex is stable, the temperature is increased above the duplex T_{M} to a temperature at which all duplex molecules become fully dissociated. The hybridization state of the duplex can be monitored through changes in signal such as those from a fluorophore and quencher pair as the duplex dissociates and plotted as a function of relative fluorescence units (RFUs) versus temperature. In a plot of the negative derivative of RFU versus temperature (i.e. a melt curve), the T_{M} may be calculated from the peak of the curve. Ideally, signals are collected at relatively small temperature increments (for example every 0.5° C.), particularly within close range of the T_{M}, to generate sufficient data points to calculate an accurate T_{M}. In some cases, reassociation analysis may be performed rather than melt analysis. In reassociation analysis, the starting temperature is one at which the duplex is dissociated and signal is measured as the temperature is lowered to a temperature at which the duplex reassociates. While this application uses the term melt analysis to describe the monitoring of the hybridization state of the duplex as the temperature changes, a person skilled in the art understands that reassociation analysis may be substituted for melt analysis in the method of the invention. Melt analysis may not always require the determination of an accurate T_{M} of a duplex. In some situations, it may only be necessary to detect signal from a target duplex at a temperature at which all target duplexes are double stranded and a temperature at which all target duplexes are single stranded. A difference in signal at these two detection temperatures indicates that the duplex has transitioned from duplex to single strand conformation at the selected temperature interval.

### V. PROBES

The methods provided herein comprise contacting a sample with at least a first high temperature probe and at least a first low temperature probe. The at least first high temperature probe hybridizes to at least a portion of the at least first target nucleic acid and produces a first signal after binding thereto, and wherein the first high temperature probe has a melting temperature that is above the melting temperatures of the primers for PCR amplification. The first low temperature probe hybridizes to at least a portion of the at least second target nucleic acid and produces a second signal after binding thereto, wherein the second signal is identical to the first signal, and wherein the at least first low temperature probe has a melting temperature that is below the melting temperatures of the primers for PCR amplification.

Several molecular probe types can be used to interrogate a target nucleic acid amplicon by melt analysis and real-time PCR. Pleiades probes (Nanogen Inc.) are comprised of a minor groove binder (MGB) and a fluorophore at the 5′-end and a non-fluorescent quencher at the 3′-end of the DNA sequence. Molecular Beacon probes are single-stranded, hairpin-shaped DNA probes that are dual-labeled with a fluorescent reporter group at the 5′ end and a fluorescent quencher group at the 3′ end. HyBeacon probes are single-stranded oligonucleotides with one or more internal base(s) labeled with a fluorescent dye. When a probe forms a duplex with its target sequence, the level of fluorescence emission increases considerably. The low temperature probe may not be designed to block exonuclease activity at the 5′ end, because it may never anneal to the amplicon during the course of the polymerase chain reaction cycling procedure. It may only anneal to one strand of the amplicon after the PCR reaction when the temperature of the reaction chamber is brought to a point that is below the Tₘ of the low temperature probe. The temperature of the reaction chamber may be slowly increased while taking optical measurements of the chamber, and the brightness of the reaction volume may decrease as the probe transitions from a mostly hybridized state (for example, stretched out) to a mostly unhybridized state (for example, random coil), the latter bringing the fluorophore and quencher pair into closer proximity which dims the output signal through Forster resonance energy transfer (FRET).

As used herein, a “probe” refers to an oligonucleotide or portions thereof, used to detect complementary target nucleic acid sequences. Probes or primers may include a detectable label. Probes may also be extended by a polymerase using a template nucleic acid. The template nucleic acid may be a partially self-complementary region of the probe or the template may be an independent partially complementary nucleic acid.

As used herein, “reporters” or “labels” are chemical or biochemical moieties useful for labeling an oligonucleotide or nucleic acid. “Reporters” or “labels” include fluorescent agents, chemiluminescent agents, chromogenic agents, quenching agents, radionuclides, enzymes, substrates, cofactors, scintillation agents, inhibitors, magnetic particles, and other moieties known in the art. “Labels” or “reporters” are capable of generating a measurable signal and may be covalently or noncovalently coupled to an oligonucleotide or nucleotide using methods known in the art.

As used herein, a “fluorescent dye” or a “fluorophore” is a chemical group that can be excited by light to emit fluorescence at a given wavelength or range of wavelengths. Dyes that may be used in the disclosed methods include, but are not limited to, fluorophores such as ALEXA FLUOR™ dyes, Fluorescein, HEX™ or AQUAPHLUOR® and others known to those skilled in the art. Examples of fluorophores include, a red fluorescent squarine dye such as 2,4-Bis [1,3,3-trimethyl-2-indolinylidenemethyl]cyclobutenediylium-1,3-dioxolate, an infrared dye such as 2,4 Bis [3,3-dimethyl-2-(1H-benz [e] indolinylidenemethyl)] cyclobutenediylium-1,3-dioxolate, or an orange fluorescent squarine dye such as 2,4-Bis [3,5-dimethyl-2-pyrrolyl]cyclobutenediylium-1,3-diololate. Additional non-limiting examples of fluorophores include quantum dots, ALEXA FLUOR® dyes (sulfonated amino-coumarin or rhodamine), aminomethylcoumarin (AMCA), BODIPY® (borondipyrromethene dye) fluorophores, including BODIPY® 630/650, BODIPY® 650/665, BODIPYR-FL, BODIPYR-R6G, BODIPY®-TMR, and BODIPY®-TRX, CASCADE BLUE® (pyrenyloxytrisulfonic acid), CYDYE™ (cyanine-based fluorophores), including but not limited to CY2™ (cyanine dye 2), CY3™ (cyanine dye 3), and CY5™ (cyanine dye 5), a DNA intercalating dye, 6-FAM™ (6-carboxyfluorescein), Fluorescein, HEX™ (hexachloro-fluorescein), 6-JOE™ (6-carboxy-4′,5′-dichloro-2′,7′-dimethoxyfluorescein), OREGON GREEN® fluorophores (fluorinated analogs of fluorescein), including OREGON GREEN® 488, OREGON GREEN® 500, and OREGON GREEN® 514, PACIFIC BLUE™ (3-carboxy-6,8-difluoro-7-hydroxycoumarin), REG™, phycobilliproteins including, but not limited to, phycoerythrin and allophycocyanin, RHODAMINE GREEN™ (CAS: 189200-71-3), RHODAMINE RED™ (a triarylmethane dye), ROX™ (carboxyrhoadmine), TAMRA™ (carboxytetramethylrhodamine), TET™ (tetrachlorofluorescein), Tetramethylrhodamine, or TEXAS RED® (sulforhodamine 101 acid chloride). A signal amplification reagent, such as tyramide (PerkinElmer), may be used to enhance the fluorescence signal.

It is contemplated that FRET-based detection systems may be used with the methods and compositions disclosed herein. FRET (fluorescence resonance energy transfer or Forster resonance energy transfer) makes use of the transfer of energy between donor and acceptor chromophores. In certain embodiments, a chromophore is attached to the hairpin sequence or the blocker and another chromophore is attached to the capture complex, such that upon attachment of the primer to the capture complex, an increase in signal will be observed by virtue of the energy transfer between the donor and acceptor chromophores. FRET-based detection reduce background and therefore allow for higher multiplexing of primer sets compared to free floating chromophore methods, particularly in closed tube and real-time detection systems.

It is also contemplated that fluorophore/quencher-based detection systems may be used with the methods and compositions disclosed herein. When a quencher and fluorophore are in proximity to each other, the quencher quenches the signal produced by the fluorophore. A conformational change in the nucleic acid molecule separates the fluorophore and quencher to allow the fluorophore to emit a fluorescent signal. Like FRET-based detection, fluorophore/quencher-based detection systems reduce background and therefore allow for higher multiplexing of primer sets compared to free floating fluorophore methods, particularly in closed tube and real-time detection systems. The oligonucleotides and nucleotides of the disclosed methods may be labeled with a quencher, suitable for quenching fluorescence of a fluorophore. Quenching may include dynamic quenching (e.g., by FRET), static quenching, or both.

Cleavable probes may be used in the instant methods. Cleavage of the cleavable probe may be achieved in a number of ways. For example, the cleavable probe may comprise one or more ribonucleotide(s). In this example, the probe hybridizes to an amplicon to create an RNA: DNA hybrid cleavage site for cleavage by RNase HII

As another example, the cleavable probe may comprise one strand of a restriction enzyme or nicking enzyme recognition sequence. Hybridization results in the formation of a cleavage site that may be recognized by the restriction enzyme or nicking enzyme for cleavage at the double stranded recognition sequence. The nicking enzyme endonuclease or restriction endonuclease is preferably thermal stable and may be engineered or naturally occurring. Double stranded cleavage, typical of restriction enzyme cleavage, or single strand cleavage of the cleavable probe may be utilized. A single stranded break may be preferable as it would allow the extended flap probe to hybridize to and cause cleavage of additional cleavable probes. Restriction enzymes may provide for single strand cleavage on cleavage sites comprising modified backbone linkages. For example, phosphorothioate (PTO), boranophosphate, methylphosphate, or peptide internucleotide linkages may be used to induce single stranded cleavage by some restriction enzymes. For example, FolI (NEB), BbsI (NEB), and BbsI (NEB) can be used with PTO. Naturally occurring nicking endonuclease cloned from Bacillus Sereothermophilus Nt.BstNBI can be used. PTO linkages can be produced by alpha thiol modified deoxynucleotides. Typically, the S isomer is employed which is incorporated into the nucleotide strand and confers nuclease resistance more effectively.

Suitable labeling schemes can be incorporated into probes to enable performing a melt analysis or for use during real-time PCR. For example, the label associated with the 5′ end of the probe may be a fluorescent reporter-quencher pair that adopts a random coil configuration in the absence of target nucleic acid. In this single stranded conformation, the quencher is in close proximity to the reporter and thus quenches reporter signal. In the presence of target nucleic acid, the random coil may adopt a linear double stranded conformation, thus separating the quencher and reporter to release the reporter from quenching. Other labeling schemes may be utilized provided they result in a probe that emits a distinguishable signal in the presence of target nucleic acid and the labeling scheme is capable of distinguishing whether the probe is in a single-stranded conformation or double-stranded monomer conformation.

TaqMan probes may be used in the instant methods. TaqMan probes rely on the 5′-3′ exonuclease activity of Taq polymerase to cleave a dual-labeled probe during hybridization to the complementary target sequence and fluorophore-based detection. As in other quantitative PCR methods, the resulting fluorescence signal permits quantitative measurements of the accumulation of the product during the exponential stages of the PCR. TaqMan probes comprise a fluorophore covalently attached to the 5′-end of the oligonucleotide probe and a quencher at the 3′-end. Several different fluorophores (e.g. 6-carboxyfluorescein, acronym: FAM, or tetrachlorofluorescein, acronym: TET) and quenchers (e.g. tetramethylrhodamine, acronym: TAMRA) are available for use. The quencher molecule quenches the fluorescence emitted by the fluorophore when excited by a thermocycler's light source via Förster resonance energy transfer (FRET). As long as the fluorophore and the quencher are in proximity, quenching inhibits any fluorescence signals. TaqMan probes are designed such that they anneal within a DNA region amplified by a specific set of primers. TaqMan probes can be conjugated to a minor groove binder (MGB) moiety (e.g., dihydrocyclopyrroloindole tripeptide) in order to increase its binding affinity to the target sequence. MGB-conjugated probes may have a higher melting temperature (Tₘ) due to increased stabilization of van der Waals forces. As the Taq polymerase extends the primer and synthesizes the nascent strand (from the single-stranded template), the 5′ to 3′ exonuclease activity of the Taq polymerase degrades the probe that has annealed to the template. Degradation of the probe releases the fluorophore from it and breaks the proximity to the quencher, thus relieving the quenching effect and allowing fluorescence of the fluorophore. Hence, fluorescence detected in the quantitative PCR thermal cycler is directly proportional to the fluorophore released and the amount of DNA template present in the PCR.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Delayed Signal Melt Probe Assay

### A. Materials and Methods

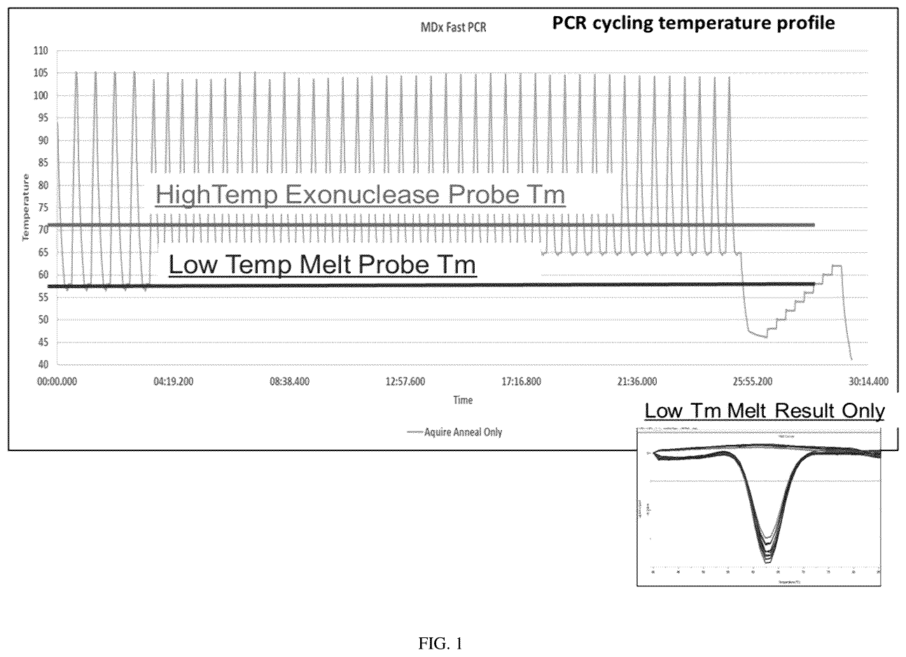

An exemplary thermocycling profile is shown in FIG. 1, which shows a temperature cycling profile of a polymerase chain reaction wherein cycles 6 through 50 oscillate between a temperature of 65° C. and 105° C. A low temperature probe with a Tₘ of 57° C. and a high temperature probe with a Tₘ of 72° C. are present in the reaction. The high temperature probe is cleaved if the high temperature probe target is present in the reaction, but the low temperature probe is not cleaved even if the target for the low temperature probe is present in the reaction. The inverse derivative of the signal from multiple reads over many temperatures is graphed and identifies the specific Tₘ of the low temperature probe, adding a confirmation in specificity. The number of measurements taken at various temperatures during the melt analysis protocol may be as low as 2, and as high as 200. A temperature below and a temperature above the Tₘ of the low temperature probe are used and may be calculated by subtracting or dividing the second signal from the first signal in order to produce a value or ratio to be compared against a preset threshold. Signal that is beyond the preset threshold will determine positivity of the target.

### Assay PCR Mastermix

A Taqman Reverse Transcription PCR mastermix was prepared containing: nuclease free water (Ambion), Diasorin Inc. propriatory MDx mastermix buffer, Assay target primers (Luminex); Flu B, RSV, Covid, Flu A, and MS2 internal PCR control, high temperature Taq-man probes (Luminex); Flu B, RSV, Covid, Flu A, and new Low-Temperature probe for detection of MS2 (Luminex). The mastermix also contained a hot-start exo-nuclease Taq enzyme, a reverse transcription enzyme, and a DNA reverse transcription blocking reagent. The exo-nuclease Taq enzyme was manufactured and obtained from Diasorin Inc. The sample target Taqman probes contained unique fluorescent dyes corresponding to 4 different fluorescent channels: Flu B-AP525, RSV-AP593, Covid-FAM, and Flu A-AP662. The low temperature probe for detection of MS2 was also labeled with AP662 and in the same fluorescent channel as Flu A. A standard exonuclease master mix with concentrations of primers and probes known in the art were used.

### Low Temperature Probe

The low temperature probe may comprise different components as long as the hybridization temperature to the amplicon, Tₘ, is below the PCR annealing temperature. The probe design used is:
MGB-AP662/GCGACAGTCACGTC/Q630 (SEQ ID NO: 1)

The low temperature probe comprises a 5′ MGB and AP662 dye, an MS2 binding region, and a 3′ Q630 quencher. The MS2 binding region has a modeled melting temperature of 55.4° C.

An exemplary high temperature probe is:
/AP-662/ccctcaaa*gccga*gatcgcgcagagact/EDQ-MGB/(SEQ ID NO: 2)

This probe was ordered from Elitech. AP-662 is a small molecule dye, * represents superbases to increase the Tₘ, and EDQ-MGB is a minor groove binder also to increase Tₘ. The viral target is Influenza A.

### PCR Cycling Protocol

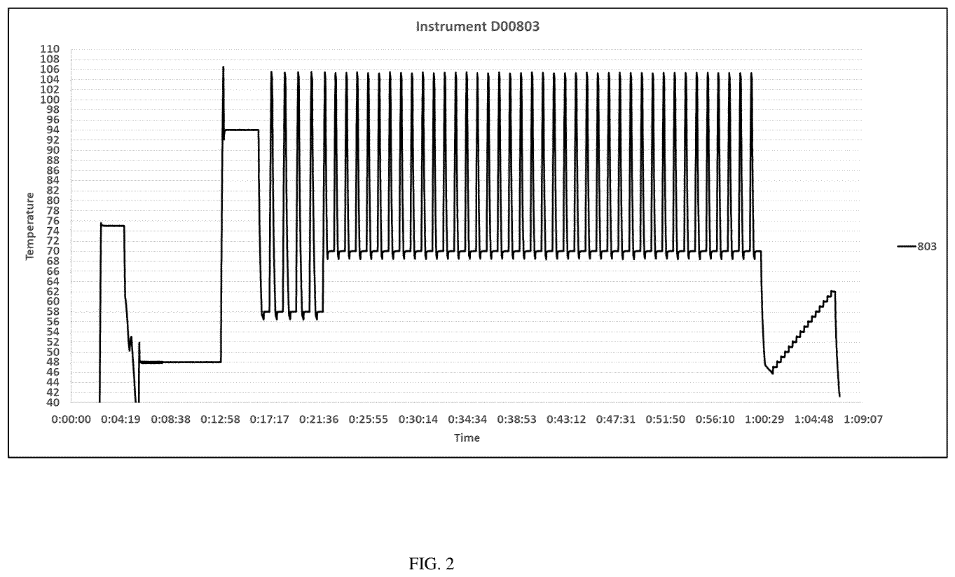

Prior to PCR cycling, the protocol contains an initial 2 min 75° C. Lysis step followed by a 5 min 48° C. hold for reverse transcription and a 2 min 94° C. hold for hot start of the DNA polymerase enzyme. The PCR cycling parameters in this experiment contain 2 stages, high delta (HD) cycling and low delta cycling (LD). The first 5 rounds of thermal cycling are high delta, with annealing temperatures at 58° C. for 30 sec holds and the instrument heated to a denature temperature of 105° C. for 1 sec hold. The remaining 40 PCR cycles are low delta with an annealing temp of 70° C. for 30 sec holds and a denature temperature of 106° C. for 1 sec hold. Post PCR cycling, a probe melt step was performed starting at 47° C. and ending at 61° C. with 1° C. increments and 20 sec holds at each step. The PCR cycling and melt profile is shown in FIG. 2. The first high delta cycles were designed to incorporate 5′nonspecific flaps of oligonucleotide sequence on each primer such that subsequent hybridization Tₘ of the primer sequences are increased, allowing for a faster PCR reaction and a higher overall annealing temperature of the PCR reaction.

### Instrument and Assay Procedure

The instrument used for assay testing was a Diasorin Liason MDX. It consists of a disk-shaped consumable for sample and test reagents, and an instrument to heat and cool the disk for PCR and take fluorescent images at specified times. In the consumable, 50 μL of the assay mastermix reagents are loaded into a reagent well and 50 μL of a sample is loaded into a corresponding sample well. The disk is sealed and loaded onto the instrument. The instrument performed the PCR cycling protocol stated above and in FIG. 2. Fluorescent readings were taken at the end of the anneal holds during the LD cycling stage and during each hold of the post PCR melt.

### B. Results

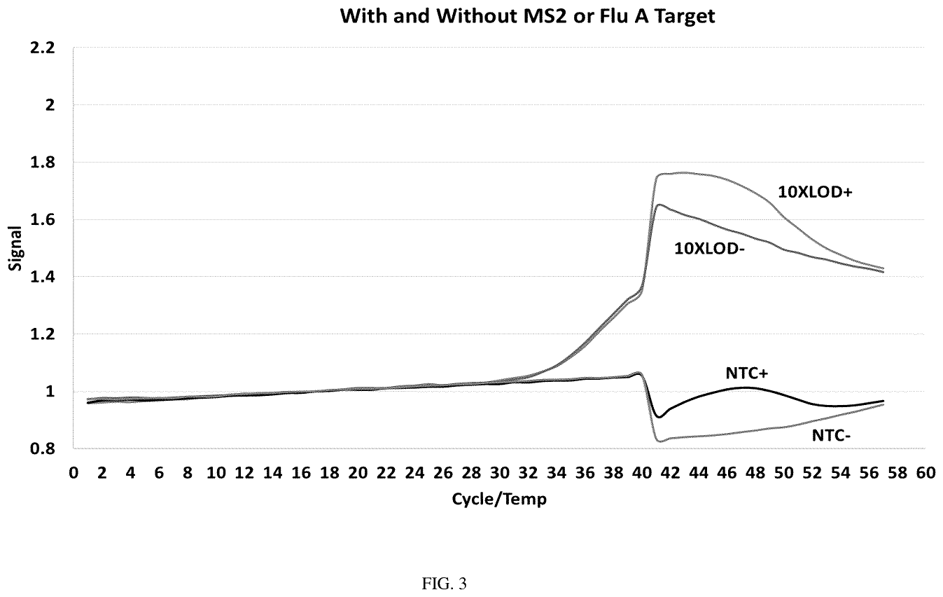

In this experiment, Flu A and MS2 were the only targets tested in the assay because they are the two targets in the same fluorescent channel. The Flu A is a Taqman probe and should only show signal increase during PCR amplification when the specific amplicon is generated and the probe is digested by the Taq polymerase. If the MS2 target is present the primers will amplify the amplicon during PCR. The low Tₘ probe for MS2 should not hybridize at the protocol annealing temperature and will not generate a signal change during PCR cycling. The Post PCR melt starts at a temperature below the MS2 probe melting temperature allowing the probe to bind if the amplicon is present. Upon heating, the probe will disassociate from the amplicon generating a signal change. The PCR data in FIG. 3 shows that if Flu A and MS2 are present or not present the positive results for either can be distinguished.

The data in FIG. 3 shows data from the 40 PCR cycles and post melt normalized to step 17. Flu A was tested at 10×LOD with and without MS2 target present. The blue line in the graph shows positive amplification during PCR representing Flu A positivity with MS2 present. In the melt post PCR, the blue line has drastic signal change around the melting temperature of the MS2 probe. The purple line on the graph shows the same 10×LOD Flu A positivity during PCR but since no MS2 target was present there is not a drastic signal change from the MS2 probe. The slight signal decrease seen may be due to dye signal temperature dependence from the digested Flu A taqman probe. The Black line in the graph is a Flu A negative with MS2 positive sample. There is no PCR amplification seen but there is a signal change during the post PCR melt indicating amplification of the MS2 amplicon. The red line in the graph shows a Flu A and MS2 negative sample, so no PCR amplification is seen and no drastic signal change during the post PCR melt. The slight signal increase seen may be due to MS2 probe random coiling opening slightly from the increase in temperature reducing the probe quenching.

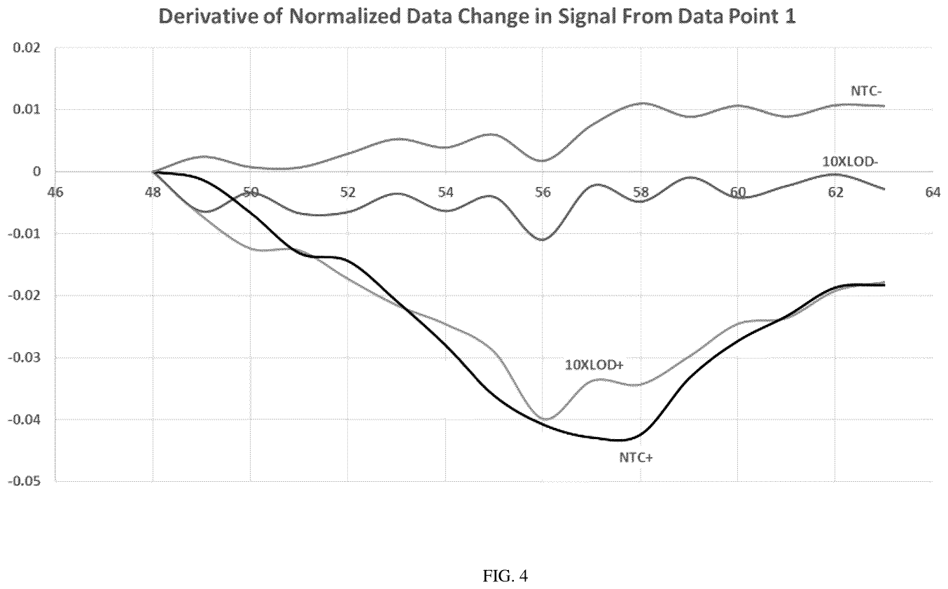

To analyze the low temperature melt probe for MS2, a derivative of the melt data can be calculated which can discern a change in signal over temperature. The maximum point of change on the derivative graph corresponds to the greatest point of inflection. This value is called the melting temperature. FIG. 4 shows the derivative of the normalized data from FIG. 3. The change in signal from the first data point is calculated to baseline all the data to 0. Without wishing to be bound by any theory, a threshold cutoff can be created and applied to the graph and peaks that are below the cutoff are positive and above the cutoff are negative. A melting temperature value corresponding to the temperature at the derivative peak can also be calculated and used as a specific cutoff. In FIG. 4, the purple and red lines, which correspond to MS2 negative samples, do not show a derivative peak compared to the positive MS2 samples.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of detecting at least two target nucleic acids in a sample, the method comprising the steps of:
a) contacting the sample with reagents comprising:
i) a first set of primers for PCR amplification of a first target nucleic acid, with a first melting temperature;
ii) a second set of primers for PCR amplification of a second target nucleic acid, with a second melting temperature;
iii) a first high temperature probe, wherein the first high temperature probe hybridizes to at least a portion of the first target nucleic acid and produces a first signal after binding thereto, and wherein the first high temperature probe has a third melting temperature that is above the first and second melting temperatures;
iv) a first low temperature probe, wherein the first low temperature probe hybridizes to at least a portion of the second target nucleic acid and produces a second signal after binding thereto, wherein the second signal is identical to the first signal, and wherein the first low temperature probe has a fourth melting temperature that is below the first and second melting temperatures;
b) amplifying the first and second target nucleic acids by a PCR reaction, wherein the PCR comprises at least two thermocycles at temperatures greater than the fourth melting temperature, and wherein the second signal is not produced during the PCR reaction;
c) detecting the first target nucleic acid by detecting the first signal from the high temperature probe during at least two thermocycles of the PCR reaction;
d) detecting the presence of the second target nucleic acid by detecting the second signal from the first low temperature probe over at least two temperatures during a post-PCR melt analysis, wherein at least one of the temperatures is below the fourth melting temperature.

2. The method of claim 1, wherein the first high temperature probe and/or the first low temperature probe comprises a fluorescent label.

3. The method of claim 2, wherein the first high temperature probe and/or the first low temperature probe comprises a fluorescent quencher.

4. The method of claim 2, wherein the first high temperature probe and/or the first low temperature probe comprises a minor groove binder.

5. The method of claim 2, wherein the first high temperature probe and/or the first low temperature probe comprises a hairpin.

6. The method of claim 2, wherein the first high temperature probe and/or the first low temperature probe comprises one or more internal nucleotide labelled fluorescent dyes.

7. The method of claim 1, wherein the first high temperature probe and/or the first low temperature probes comprise(s) a cleavable probe.

8. The method of claim 2, wherein the first and/or second signals from the first high temperature probe and/or first low temperature probe is/are fluorescent signal(s).

9. The method of claim 1, wherein the first high temperature probe is a TaqMan probe.

10. The method of claim 1, wherein the PCR reaction amplifies a short fragment of the first and second nucleic acids.

11. The method of claim 1, wherein the PCR reaction is completed in less than 20 minutes.

12. The method of claim 1, wherein the PCR reaction comprises a high delta cycling phase and a low delta cycling phase.

13. The method of claim 12, wherein the high delta cycling phase comprises annealing temperatures at 58° C. for 30 second holds, and denaturing temperatures at 105° C. for 1 second holds.

14. The method of claim 13, wherein the low delta cycling phase comprises annealing temperatures at 70° C. for 30 second holds, and denaturing temperatures at 106° C. for 1 second holds.

15. The method of claim 1, wherein the post-PCR melt analysis comprises thermocycling steps starting at 47° C. and ending at 61° C. with 1° C. increments and 20 second holds at each step.

16. The method of claim 1, wherein the post-PCR melt analysis comprises thermocycling steps with at least one temperature is below the fourth melting temperature, and at least one temperature is above the fourth melting temperature.

17. The method of claim 1, wherein the sample further comprises a third and fourth target nucleic acid, and wherein step a) further comprises contacting the sample with:
v) a third set of primers for PCR amplification of the third target nucleic acid, with a fifth melting temperature;
vi) a fourth set of primers for PCR amplification of the fourth target nucleic acid, with a sixth melting temperature;
vii) a second high temperature probe wherein the second high temperature probe hybridizes to at least a portion of the third target nucleic acid and produces a third signal, and wherein the second high temperature probe has a seventh melting temperature that is above the first, second, fifth, and sixth melting temperatures;
viii) a second low temperature probe, wherein the second low temperature probe hybridizes to at least a portion of the fourth target nucleic acid and produces a fourth signal, wherein the fourth signal is identical to the third signal, and wherein the second low temperature probe has a eighth melting temperature that is below the first, second, fifth, and sixth melting temperatures;
and wherein step b) further comprises amplifying the third and fourth target nucleic acids by a PCR reaction, wherein the PCR comprises at least two thermocycles at temperatures greater than the fourth and eighth melting temperatures, and wherein the second and fourth signals are not produced during the PCR reaction;
and wherein step c) further comprises detecting the first and third target nucleic acids by detecting the first and third signals from the first and second high temperature probes during at least two thermocycles of the PCR reaction;
and wherein step d) further comprises detecting the presence of the second and fourth target nucleic acids by detecting the second and fourth signals from the first and second low temperature probes over at least two temperatures during a post-PCR melt analysis, wherein at least one of the temperatures is below the fourth and eighth melting temperatures.

18. The method of claim 17, wherein the second high temperature probe and/or the second low temperature probe comprises a fluorescent label.

19. The method of claim 18, wherein the second high temperature probe and/or the second low temperature probe comprise a fluorescent quencher.

20. The method of claim 17, wherein the second high temperature probe and/or the second low temperature probe comprises a minor groove binder.

21. The method of claim 17, wherein the second high temperature probe and/or the second low temperature probe comprises a hairpin.

22. The method of claim 17, wherein the second high temperature probe and/or the second low temperature probe comprises one or more internal nucleotide labelled fluorescent dyes.

23. The method of claim 17, wherein the second and/or third signals from the second high temperature probe and/or second low temperature probe is/are fluorescent signal(s).

24. The method of claim 17, wherein the second high temperature probe and/or the second low temperature probes comprise(s) a cleavable probe.

25. The method of claim 17, wherein the second high temperature probe is a TaqMan probe.

26. The method of claim 17, wherein the PCR reaction amplifies a short fragment of the second and third nucleic acids.

27. The method of claim 17, wherein the PCR reaction is completed in less than 20 minutes.

28. A kit comprising:
a) a first set of primers for PCR amplification of a first target nucleic acid, with a first melting temperature;
b) a second set of primers for PCR amplification of a second target nucleic acid, with a second melting temperature;
c) a first high temperature probe, wherein the first high temperature probe hybridizes to at least a portion of the first target nucleic acid and produces a first signal, and wherein the first high temperature probe has a third melting temperature that is above the first and second melting temperatures;
d) a first low temperature probe, wherein the first low temperature probe hybridizes to at least a portion of the second target nucleic acid and produces a second signal, wherein the second signal is identical to the first signal, and wherein the first low temperature probe has a fourth melting temperature that is below the first and second melting temperatures.

29. A composition comprising:
a) a first high temperature probe, wherein the first high temperature probe hybridizes to at least a portion of a first target nucleic acid and produces a first signal, and wherein the first high temperature probe has a first melting temperature;
b) a first low temperature probe, wherein the first low temperature probe hybridizes to at least a portion of a second target nucleic acid and produces a second signal, wherein the second signal is identical to the first signal, and wherein the first low temperature probe has a second melting temperature that is below the first melting temperature.


SEQUENCE LISTING (2 sequences)

SEQ ID NO: 1 - DNA, 14 bp, synthetic construct
  source 1..14: mol_type = other DNA
gcgacagtcacgtc

SEQ ID NO: 2 - DNA, 28 bp, synthetic construct
  source 1..28: mol_type = other DNA
ccctcaaagccgagatcgcgcagagact